(19) United States
(12) Patent Application Publication
(10) Pub. No.: US 2012/0301549 A1
(43) Pub. Date: Nov. 29, 2012

(54) COMPLEX FORMULATION COMPRISING ASPIRIN COATED WITH BARRIER CONTAINING HYDROPHOBIC ADDITIVE, AND HMG-COA REDUCTASE INHIBITOR

(75) Inventors: Jong Soo Woo, Suwon-si (KR); Jae Hyun Park, Suwon-si (KR); Yong Il Kim, Suwon-si (KR); Young Jun Na, Suwon-si (KR); Jun Young Choi, Suwon-si (KR); Yun Ah Lee, Bucheon-si (KR)

(73) Assignee: HANMI SCIENCE CO., LTD., Hwaseong-si, Gyeonggi-do (KR)

(21) Appl. No.: 13/576,585

(22) PCT Filed: Jan. 26, 2011
(86) PCT No.: PCT/KR2011/000541

§ 371 Date: Aug. 1, 2012

(30) Foreign Application Priority Data
Feb. 2, 2010 (KR) 10-2010-0009636

(51) Int. Cl. A61K 9/50; A61P 9/00; A61K 31/60

(52) U.S. Cl. 424/498; 514/165; 514/161; 514/163

(57) ABSTRACT

Provided is a complex formulation for the prevention or treatment of cardiovascular diseases, comprising: a) aspirin coated with a barrier containing a hydrophobic additive; and b) an HMG-CoA reductase inhibitor, which has improved storage stability by preventing the deterioration in the stability of HMG-CoA reductase which is caused by salicylic acid, thereby being used in the treatment of hypertension and hypercholesterolemia.

### FIELD OF THE INVENTION

The present invention relates to a complex formulation for the prevention or treatment of cardiovascular diseases, which comprises a) aspirin coated with a barrier containing a hydrophobic additive, and b) an HMG-CoA reductase inhibitor.

### BACKGROUND OF THE INVENTION

Hyperlipidemia is the condition of abnormally elevated levels of lipids such as cholesterols, triglycerides, and others, in the plasma. Hyperlipidemia, particularly hypercholesterolemia, induces arterial thrombosis, resulting in arteriosclerosis which is thick accumulation of lipids within the blood vessel. It is clinically important since it contributes to cardiovascular diseases such as ischemic heart disease, angina pectoris, and myocardial infarction. The prevention of arteriosclerosis may be achievable by way of the treatment of hypercholesterolemia highly associated therewith.

For decades, HMG-CoA reductase inhibitors have been used to treat hyperlipidemia. Such compounds have been known to lower total cholesterol and LDL-cholesterol in human body and to elevate HDL-cholesterol in some individuals. They inhibit HMG-CoA reductase involved in the conversion of HMG-CoA to mevalonate, which is an early and rate-limiting step in the biosynthesis of cholesterol. This process increases receptors of LDLs which induce arteriosclerosis, leading to the decrease in the concentration of LDL within blood (Grundy S. M., N Engl. J. Med., 319(1):24-32, 25-26, 31 (1988). Examples of HMG-CoA reductase inhibitors include mevastatin (U.S. Pat. No. 3,983,140), lovastatin (also called mevinolin; U.S. Pat. No. 4,231,227), pravastatin (U.S. Pat. Nos. 4,346,227 and 4,410,629), pravastatin lactone (U.S. Pat. No. 4,448,979), velostatin (also called synvinolin; U.S. Pat. Nos. 4,448,784 and 4,450,171), simvastatin, rivastatin, fluvastatin, atorvastatin, rosuvastatin, cerivastain, and others.

Another mechanism leading to arteriosclerosis is thrombus formation. Thrombus is achieved via the interaction of platelets and plasma coagulation factor in an injured vessel, which also induces arteriosclerosis. Aspirin is used as an antipyretic to reduce fever, as an analgesic to relieve minor aches and pains, and as an agent to prevent arterial thrombosis. Aspirin (also known as acetylsalicylic acid) irreversibly acetylates cyclooxygenase, thereby inhibiting the production of thromboxane A2 (TXA2), which is synthesized by platelets to promote platelet aggregation. This blocks platelet aggregation in blood to decrease platelets.

The combination of an HMG-CoA reductase inhibitor and aspirin may be useful in treating various cardiovascular diseases by providing each effect of the drugs at the same time, as well as in treating a cardiovascular disease effectively by providing synergistic effects of the drugs. In addition, the complex formulation comprising the combination of said drugs may allow for greater ease of administration than the individual administrations of said drugs.

HMG-CoA reductase inhibitors exhibit poor bioavailability and are absorbed in the gastrointestinal tract, and thus it would be beneficial to be rapidly released in the gastrointestinal tract. Meanwhile, aspirin may exhibit adverse side effects, e.g., gastric ulcer or gastric bleeding when it is released within the gastrointestinal tract, and may interact adversely with HMG-CoA reductase inhibitors when both are released at the same time within the gastrointestinal tract. Thus, it needs to render aspirin to be released within not the stomach but the small intestine. The present inventors have filed an application for a formulation comprising an aspirin-containing granule and an HMG-CoA reductase inhibitor-containing granule (Korean Application Publication No. 2009-0030452). However, during storage, aspirin in the formulation degrades into salicylic acid by hydrolysis, and the resulting salicylic acid may degrade HMG-CoA reductase inhibitors which are unstable under acidic conditions.

The present inventors have found that the deterioration in the stability of HMG-CoA reductase caused by salicylic acid can be prevented by coating aspirin with a barrier containing a hydrophobic additive.

### SUMMARY OF THE INVENTION

Therefore, it is an object of the present invention to provide a complex formulation for the prevention or treatment of cardiovascular diseases, which has improved storage stability by preventing the deterioration in the stability of HMG-CoA reductase inhibitors which is caused by salicylic acid.

In accordance with one aspect of the present invention, there is provided a complex formulation for the prevention or treatment of cardiovascular diseases, comprising: a) aspirin coated with a barrier containing a hydrophobic additive; and b) an HMG-CoA reductase inhibitor. Preferably, the barrier comprises the hydrophobic additive in an amount of 3.8-60% by weight based on the total weight of the barrier.

The complex formulation of the present invention exhibits excellent effect on the prevention and treatment of cardiovascular diseases, exerting improved storage stability by preventing the deterioration in the stability of HMG-CoA reductase inhibitors which is caused by salicylic acid, thereby being useful for the prevention and treatment of cardiovascular diseases.

### BRIEF DESCRIPTION OF THE DRAWINGS

The above and other objects and features of the present invention will become apparent from the following description of the invention, when taken in conjunction with the accompanying drawings, which respectively show:

FIG. 1: the stability test of the complex formulation of the present invention after 4 months under accelerated conditions, showing the amounts of atorvastatin lactone and salicylic acid;

FIG. 2: the stability test of the complex formulation of the present invention after 4 months under accelerated conditions, showing the amounts of rosuvastatin lactone and salicylic acid;

FIG. 3: the stability test of the complex formulation of the present invention after 4 months under accelerated conditions depending on the amount of a hydrophobic additive, showing the amounts of atorvastatin lactone and salicylic acid;

FIG. 4: the dissolution rates of coated aspirin pellets of Examples 2 and 4, ‘Aspirin Protect®’, and ‘Astrix®’ for 1 hour depending on pH; and

FIG. 5: the change of dissolution rate of the complex formulation of the present invention for 1 hour depending on the amount of a hydrophobic additive.

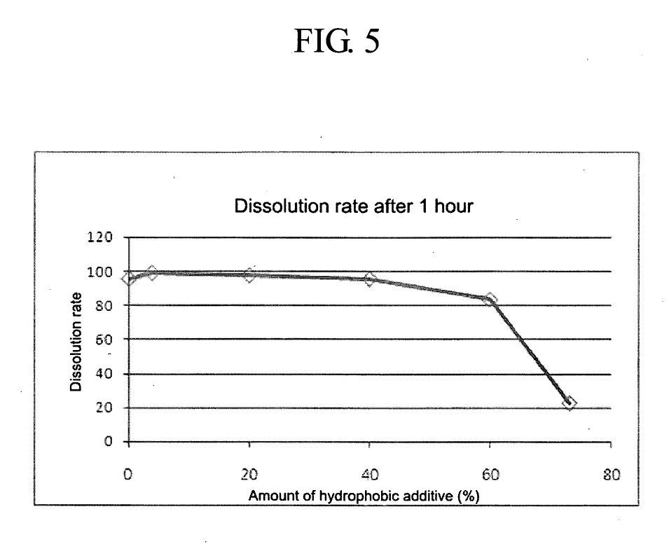

### DETAILED DESCRIPTION OF THE INVENTION

The present invention provides a complex formulation for the prevention or treatment of cardiovascular diseases, comprising: a) aspirin coated with a barrier containing about 3.8˜60% of a hydrophobic additive by weight based on the total amount of the barrier, as a 1^{st} pharmacologically active ingredient; and b) an HMG-CoA reductase inhibitor as a 2^{nd} pharmacologically active ingredient. Hereinafter, described are the properties and kinds of each ingredient consisting of the complex formulation of the present invention.

(i) 1^{st} Pharmacologically Active Ingredient (Aspirin)

Aspirin is used as 1^{st} pharmacologically active ingredient in the present invention so as to prevent and treat arterial thrombosis by blocking the platelet aggregation in blood. It may be employed in an amount of 10 mg to 2 g per the formulation and in the faun of pellets or granules.

(ii) Hydrophobic Additive (Coating Base)

A hydrophobic additive is used in the present invention so as to block the migration of salicylic acids into the granule layer containing an HMG-CoA reductase inhibitor, the use of which is different to enteric coating bases for releasing drugs depending on pH. When aspirin is coated with conventional coating bases, water-soluble and hydrophilic aspirin or salicylic acid derived from aspirin migrates to the coating layer, consequently penetrating the layer. But, when a hydrophobic additive is added to the coating layer, it is possible to prevent the migration of the drug to the coating layer and the consequent adverse influence of said drug on HMC-CoA reductase inhibitors. Such hydrophobic additive is a coating base which is irrelevant to pH and is not used for sustained- or delayed-release.

Examples of hydrophobic additives include waxes such as carnauba wax, glyceryl monostearate, glyceryl monooleate and beeswax; and synthetic or semi-synthetic hydrophobic polymers such as ethyl cellulose, aminoalkyl methacrylate copolymer RS, ethyl acrylate-methyl methacrylate copolymer, polyvinyl chloride, polyvinyl acetate and cellulose acetate.

The barrier containing such hydrophobic additive may further comprise a plasticizer such as triethyl citrate, polyethylene glycol, propylene glycol, acetylated monoglyceride, diethyl phthalate and dibutyl sebacate, and may also comprise additional coating bases commonly used in pharmaceutical industry such as HPMC, HPC, polyvinyl alcohol, and others. In addition, talc, titanium dioxide, and others may be used to prevent the adhesion of pellets during coating procedure.

The hydrophobic additive may be used in an amount of about 3.8% or more by weight based on the total amount of the barrier, and it is preferred not to exceed about 60% by weight. When the amount exceeds 60% by weight based on the total amount of the barrier, the release of drug would be excessively delayed.

(iii) 2^{nd} Pharmacologically Active Ingredient (HMG-CoA Reductase Inhibitor)

An HMG-CoA reductase inhibitor is used as a 2^{nd} pharmacologically active ingredient so as to prevent or treat hyperlipidemia and arteriosclerosis by lowering the concentration of lipoproteins or lipids. Examples of HMG-CoA reductase inhibitors include mevastatin, rosuvastatin, atorvastatin, lovastatin, pravastatin, pravastatin lactone, pitavastatin, bervastatin, velostatin, simvastatin, rivastatin, fluvastatin, cerivastatin or isomers or salts and combinations thereof.

The HMG-CoA reductase inhibitor may be employed in an amount of 5 mg to 80 mg per the formulation and in the form of granules or pellets.

(iv) Enteric Coating Base

The complex formulation of the present invention may further comprise an enteric coating layer between the aspirin core and the hydrophobic barrier. The enteric coating base is not used for the purpose of preventing the interaction of the released salicylic acid and HMG-CoA reductase inhibitors. The effects of enteric coating base are not enough to inhibit the action of released salicylic acid as evidenced by the experiments of the present invention. The main objective of use of the enteric coating base is to allow aspirin to be released into not the stomach of a low pH but the small intestine, particularly the upper small intestine, of high pH. Examples of enteric coating bases include hydroxypropyl methylcellulose phthalate, cellulose acetate phthalate, methacrylic acid copolymer, and hydroxypropyl methylcellulose acetate succinate.

The coating base may be used in a weight ratio of 0.1 to 0.5 based on 1 weight of the core.

The complex formulation of the present invention may further comprise a stabilizing agent for enhancement stability of HMG-CoA reductase inhibitor, and examples of stabilizing agents include antioxidants such as tocopherol, butylated hydroxytoluene (BHT), butylated hydroxyanisole (BHA), ascorbic acid and erythorbic acid; minerals such as CaCO₃, MgCO₃, NaHCO₃, KH₂PO₄ and K₂HPO₃; basic additives such as meglumine, arginine and glycine; and other stabilizing agents such as organic acids, e.g., citric acid and fumaric acid, or salts thereof.

The complex formulation of the present invention may be prepared by the method comprising: (1) preparing an aspirin granule or pellet which is coated with a barrier containing a hydrophobic additive; (2) preparing an HMG-CoA reductase inhibitor granule or pellet; and (3) filling a capsule with the aspirin granule or pellet and the HMG-CoA reductase inhibitor granule or pellet, prepared in steps (1) and (2), or compressing said granules or pellets.

Each process in the preparation of the complex formulation of the present invention may be carried out in accordance with conventional processes known in the pharmaceutical industry. The average diameter of the aspirin granule or pellet prepared in step (1) is preferably 1,200 μm or less, more preferably 1,000 μm or less. When the pellet size is bigger than 1,200 μm, the mixing degree between both pharmacologically active ingredients become poor, which may adversely affect the homogeneity of the complex formulation in a form of tablets.

The present invention also provides a method of preventing or treating cardiovascular diseases, which comprises administering the complex formulation of the present invention to a mammal in need thereof.

The complex formulation of the present invention may be administered orally as an active ingredient in an effective amount ranging from about 0.01 to 100 mg/kg, preferably 0.2 to 50 mg/kg body weight per day in case of mammals including human in a single dose or in divided doses. The dosage of the active ingredient may be adjusted in light of various relevant factors such as the condition of the subject to be treated, type and seriousness of illness, administration rate, and opinion of doctor. In certain cases, an amount less than the above dosage may be suitable. An amount greater than the above dosage may be used unless it causes deleterious side effects and such amount can be administered in divided doses per day.

The following Examples are given for the purpose of illustration only, and are not intended to limit the scope of the invention.

### Example 1

### Preparation of Aspirin Pellet and Granule

<1-1> Preparation of Aspirin Pellet

In accordance with Table 1, aspirin (Spectrum Chemical, US), hydroxypropyl methylcellulose (HPMC; Shinetsu, Japan), citric acid and talc (Nippon talc, Japan) were dissolved and dispersed in a mixed solution of water and ethanol to prepare an aspirin-containing coating solution. The coating solution was sprayed while fluidizing microcrystalline spherical beads (cellet; Phainiatrans) using a fluidized bed spray coater (NQ-125, Fuji Paudal, Japan), to prepare an aspirin pellet.

<1-2> Preparation of Aspirin Granule

In accordance with Table 1, aspirin, Avicel (FMC biopolymer, US), citric acid and mannitol were mixed and kneaded using hydroxypropyl methylcellulose (HPC) dissolved in water and ethanol, followed by extrusion using an extruder (MG-55, Dalton, Japan). Then, the extruded product was spheronized using a spheronizer (Q-230T, Dalton, Japan) to obtain a spherical aspirin granule.

TABLE 1

| Ingredients | <1-1> Aspirin pellet | <1-2> Aspirin granule |
|---|---|---|
| Aspirin | 100 mg | 100 mg |
| Cellet | 29 mg | — |
| Citric acid | 10 mg | 10 mg |
| HPMC | 10 mg | — |
| Talc | 1 mg | — |
| Avicel | — | 28 mg |
| Mannitol | — | 60 mg |
| HPC | — | 2 mg |
| <Water> | <100> | <30> |
| <Ethanol> | <500> | <10> |
| Total weight | 150 mg | 200 mg |

### Examples 2 to 7

### Coating with Barrier Containing Hydrophobic Additive (1)

In accordance with Table 2, the aspirin pellet or granule of Example 1 was coated with barriers containing various compositions of hydrophobic additives. Specifically, HPMC, acetylated monoglyceride (myvacet®; Kerry bio-science, US), talc, titanium dioxide (TiO₂), and hydrophobic additives (carnauba wax or ethyl cellulose (EC, Colorcon)) were dissolved and dispersed in a mixed solution of water and ethanol to prepare barrier coating solutions. Then, each coating solution was sprayed while fluidizing the aspirin pellet or granule (cellet; Pharmatrans) using a fluidized bed spray coater (NQ-125, Fuji Paudal, Japan), to prepare barrier-coated aspirin pellets.

TABLE 2

| Ingredients | Example 2 | Example 3 | Example 4 | Example 5 | Example 6 | Example 7 |
|---|---|---|---|---|---|---|
| Example <1-1> | 150 mg | 150 mg | 150 mg | 150 mg | 150 g | — |
| Example <1-2> | — | — | — | — | — | 200 mg |
| HPMC | 20 mg | 10 mg | 10 mg | 10 mg | 10 mg | 10 mg |
| Carnauba wax | 1 mg | 3 mg | 8 mg | 18 mg | — | 8 mg |
| EC | — | — | — | — | 8 mg | — |
| Myvacet ® | 2 mg | 1 mg | 1 mg | 1 mg | 1 mg | 1 mg |
| Talc | 1 mg | 0.5 mg | 0.5 mg | 0.5 mg | 0.5 mg | 0.5 mg |
| Titanium dioxide | 2 mg | 0.5 mg | 0.5 mg | 0.5 mg | 0.5 mg | 0.5 mg |
| <Water> | <50> | <50> | <50> | <50> | <50> | <50> |
| <Ethanol> | <200> | <200> | <200> | <200> | <200> | <200> |
| Total weight | 176 mg | 165 mg | 170 mg | 177 mg | 170 mg | 220 mg |
| Hydrophobic | 3.8% | 20.0% | 40.0% | 60.0% | 40.0% | 40.0% |
| additive (%) | | | | | | |
| in coating bases | | | | | | |

### Comparative Examples 1 to 3

### Conventional Barrier or Enteric Coating

In order to prepare pellets or granules having a conventional barrier or enteric coating, the aspirin pellet or granule of Example 1 was coated with coating solutions shown in Table 3. Specifically, HPMC or HPMCP (hydroxypropyl methylcellulose phthalate; Shinetsu, Japan), Myvacet®, talc and titanium dioxide were dissolved and dispersed in a mixed solution of water and ethanol or acetone, to prepare barrier coating solutions. Then, the each coating solution was sprayed while fluidizing the aspirin pellet or granule of Example 1 using a fluidized bed spray coater (NQ-125, Fuji Paudal, Japan), to prepare conventional barrier-coated or enteric-coated aspirin pellets or granules.

TABLE 3

| | Comparative | Comparative | Comparative |
|---|---|---|---|
| Ingredients | Example 1 | Example 2 | Example 3 |
| Example <1-1> | 150 mg | — | 150 mg |
| Example <1-2> | — | 200 mg | — |
| HPMC | 20 mg | 20 mg | — |
| HPMCP | — | — | 40 mg |
| Myvacet ® | 2 mg | 2 mg | 2 mg |
| Talc | 1 mg | 1 mg | 1 mg |
| Titanium | 2 mg | 2 mg | 2 mg |
| dioxide | | | |
| <Water> | <50> | <50> | — |
| <Ethanol> | <200> | <200> | <100> |
| <Acetone> | — | — | <300> |
| Total weight | 175 mg | 225 mg | 195 mg |

### Example 8 and Comparative Example 4

### Coating with Barrier Containing Hydrophobic Additive (2)

The pellets of Example <1-1> and Comparative Example 3 were coated with barriers containing a hydrophobic additive as shown in Table 4, respectively, according to same method with Examples 2 to 7.

TABLE 4

| | | Comparative |
|---|---|---|
| Ingredients | Example 8 | Example 4 |
| Comparative Example 3 | 195 mg | — |
| Example <1-1> | — | 150 mg |
| HPMC | 10 mg | 10 mg |
| Carnauba wax | 8 mg | 30 mg |
| Myvacet ® | 1 mg | 1 mg |
| Talc | 0.5 mg | 0.5 mg |
| Titanium dioxide | 0.5 mg | 0.5 mg |
| <Water> | <50> | <50> |
| <Ethanol> | <200> | <200> |
| Total | 215 mg | 192 mg |
| Hydrophobic additive in | 40.0% | 73.2% |
| coating bases (%) | | |

### Example 9

### Preparation of HMG-CoA reductase inhibitor granule

Granules containing various HMG-CoA reductase inhibitors were prepared.

<9-1> Preparation of Atorvastatin-Containing Granule

In accordance with Table 5, atorvastatin calcium (TEVA, Israel) was mixed with Avicel®, croscarmellose sodium (DMV international), lactose (DMV international) and magnesium carbonate, and kneaded with a binding solution in which HPC and polysorbate 80 are dissolved in water. The combined product was dried, and sieved through a 30 mesh sieve to prepare an atorvastatin-containing granule.

<9-2> Preparation of Rosuvastatin-Containing Granule

The procedure of Example <9-1> was repeated except for using rosuvastatin calcium (MSN, India) instead of atorvastatin calcium to obtain a rosuvastatin-containing granule.

TABLE 5

| Ingredients | Example <9-1> | Example <9-2> |
|---|---|---|
| Atorvastatin | 10.4 mg | — |
| Rosuvastatin | — | 10.4 mg |
| Avicel ® | 15 mg | 15 mg |
| Lactose | 40 mg | 40 mg |
| Magnesium carbonate | 20 mg | 20 mg |
| Croscarmellose sodium | 6 mg | 6 mg |
| <Water> | <25> | <25> |
| Total weight | 110.4 mg | 110.4 mg |

### Formulation Examples 1 to 8 and Comparative Formulation Examples 1 to 7

### Preparation of Complex Formulation Containing Aspirin/HMG-CoA Reductase Inhibitor

By the combinations of Examples and Comparative Examples above, complex formulations containing aspirin and HMG-CoA reductase inhibitor were prepared in accordance with Table 6. Specifically, granules (or pellets) corresponding to aspirin 100 mg and HMG-CoA reductase inhibitor 10 mg, respectively, were filled into size #0 capsules to obtain complex formulations. Comparative Formulation Examples 5 and 6 are formulations prepared by filling only HMC-CoA reductase inhibitors into size #0 capsules.

TABLE 6

| Ingredients | Aspirin | Atorvastatin | Rosuvastatin |
|---|---|---|---|
| Formul. Ex. 1 | Ex. 2 | Ex. <9-1> | |
| Formul. Ex. 2 | Ex. 3 | Ex. <9-1> | |
| Formul. Ex. 3 | Ex. 4 | Ex. <9-1> | |
| Formul. Ex. 4 | Ex. 5 | Ex. <9-1> | |
| Formul. Ex. 5 | Ex. 6 | Ex. <9-1> | |
| Formul. Ex. 6 | Ex. 7 | Ex. <9-1> | |
| Formul. Ex. 7 | Ex. 8 | Ex. <9-1> | |
| Formul. Ex. 8 | Ex. 8 | | Ex. <9-2> |
| Comp. Formul. Ex. 1 | Comp. Ex. 1 | Ex. <9-1> | |
| Comp. Formul. Ex. 2 | Comp. Ex. 2 | Ex. <9-1> | |
| Comp. Formul. Ex. 3 | Comp. Ex. 3 | Ex. <9-1> | |
| Comp. Formul. Ex. 4 | Comp. Ex. 3 | | Ex. <9-2> |
| Comp. Formul. Ex. 5 | | Ex. <9-1> | |
| Comp. Formul. Ex. 6 | | | Ex. <9-2> |
| Comp. Formul. Ex. 7 | Ex. <1-1> | Ex. <9-1> | |

### Experimental Example 1

### Stability Test of Formulations

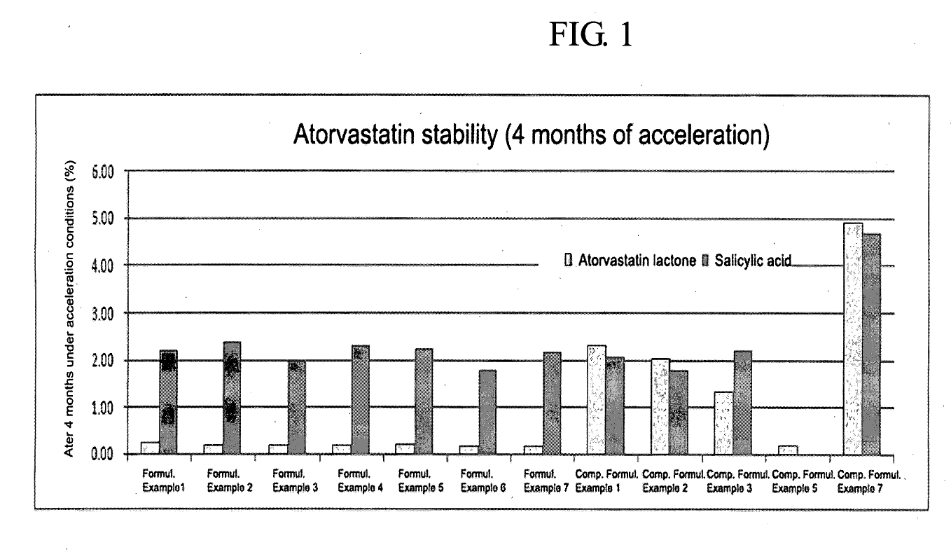

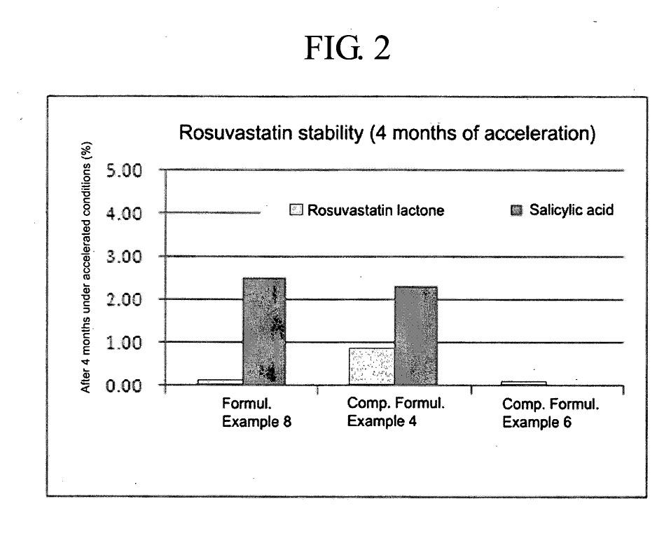

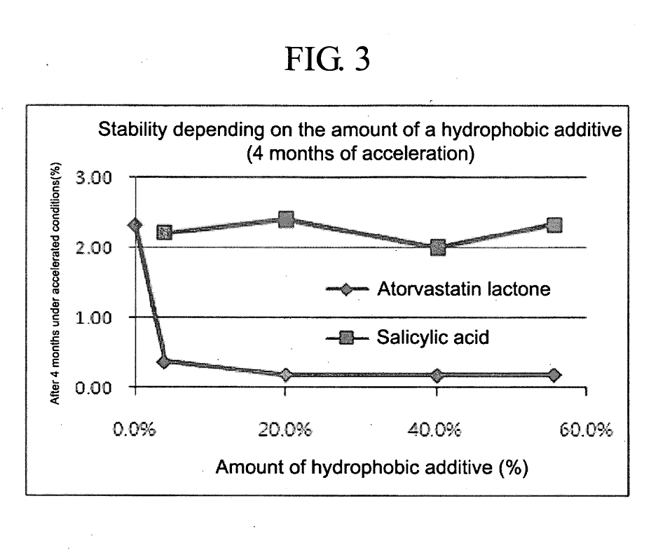

The complex formulations prepared from Formulation Examples 1 to 8 and Comparative Formulation Examples 1 to 7 were each packaged together with 1 g of silica gel in an HDPE bottle and tested for their stability for 2 and 4 months by storing under accelerated conditions (45° C., 75% RH). In case of aspirin, the content of released salicylic acid was measured in accordance with the USP (United States Pharmacopeia) specification for ‘aspirin tablet’ and ‘aspirin delayed-release capsule’. In case of atorvastatin, the contents of a representative acid hydrolyzate, i.e., atorvastatin lactone and total related compounds were measured. In case of rosuvastatin, the contents of rosuvastatin lactone and total related compounds were measured. The results are shown in Tables 7 and 8, and FIGS. 1 to 3. FIGS. 1 and 2 are graphs showing the stabilities after 4 months under accelerated conditions, and FIG. 3 is a graph showing the stability after 4 months under accelerated conditions depending on the amount of a hydrophobic additive.

TABLE 7

| | | | Total related | |
|---|---|---|---|---|
| | | | compounds | |
| | | | of | Salicylic |
| | | Atorvastatin | atorvastatin | acid |
| | | lactone (%) | (%) | (%) |
| Formul. Ex. 1 | Initial | 0.10 | 0.13 | 0.16 |
| | Two months of | 0.15 | 0.23 | 1.43 |
| | acceleration | | | |
| | Four months of | 0.25 | 0.40 | 2.21 |
| | acceleration | | | |
| Formul. Ex. 2 | Initial | 0.10 | 0.14 | 0.27 |
| | Two months of | 0.14 | 0.21 | 1.53 |
| | acceleration | | | |
| | Four months of | 0.19 | 0.31 | 2.40 |
| | acceleration | | | |
| Formul. Ex. 3 | Initial | 0.10 | 0.13 | 0.29 |
| | Two months of | 0.14 | 0.21 | 1.56 |
| | acceleration | | | |
| | Four months of | 0.18 | 0.29 | 2.00 |
| | acceleration | | | |
| Formul. Ex. 4 | Initial | 0.10 | 0.14 | 0.31 |
| | Two months of | 0.13 | 0.20 | 1.47 |
| | acceleration | | | |
| | Four months of | 0.19 | 0.30 | 2.32 |
| | acceleration | | | |
| Formul. Ex. 5 | Initial | 0.10 | 0.13 | 0.37 |
| | Two months of | 0.18 | 0.27 | 1.49 |
| | acceleration | | | |
| | Four months of | 0.22 | 0.35 | 2.24 |
| | acceleration | | | |
| Formul. Ex. 6 | Initial | 0.10 | 0.14 | 0.27 |
| | Two months of | 0.15 | 0.23 | 1.40 |
| | acceleration | | | |
| | Four months of | 0.19 | 0.30 | 1.80 |
| | acceleration | | | |
| Formul. Ex. 7 | Initial | 0.10 | 0.13 | 0.27 |
| | Two months of | 0.14 | 0.21 | 1.58 |
| | acceleration | | | |
| | Four months of | 0.19 | 0.33 | 2.19 |
| | acceleration | | | |
| Comp. Formul. | Initial | 0.11 | 0.23 | 0.21 |
| Ex. 1 | Two months of | 0.82 | 2.52 | 1.67 |
| | acceleration | | | |
| | Four months of | 2.32 | 3.55 | 2.08 |
| | acceleration | | | |
| Comp. Formul. | Initial | 0.10 | 0.22 | 0.25 |
| Ex. 2 | Two months of | 0.64 | 1.15 | 1.20 |
| | acceleration | | | |
| | Four months of | 2.05 | 2.75 | 1.80 |
| | acceleration | | | |
| Comp. Formul. | Initial | 0.10 | 0.21 | 0.38 |
| Ex. 3 | Two months of | 0.45 | 0.65 | 1.65 |
| | acceleration | | | |
| | Four months of | 1.35 | 2.14 | 2.20 |
| | acceleration | | | |
| Comp. Formul. | Initial | 0.11 | 0.13 | |
| Ex. 5 | Two months of | 0.15 | 0.21 | |
| | acceleration | | | |
| | Four months of | 0.19 | 0.29 | |
| | acceleration | | | |
| Comp. Formul. | Initial | 0.11 | 0.15 | 0.29 |
| Ex. 7 | Two months of | 1.90 | 3.50 | 2.40 |
| | acceleration | | | |
| | Four months of | 4.90 | 6.20 | 4.70 |
| | acceleration | | | |

TABLE 8

| | | | Total related | |
|---|---|---|---|---|
| | | | compounds | |
| | | | of | Salicylic |
| | | Rosuvastatin | rosuvastatin | acid |
| | | (lactone, %) | (%) | (%) |
| Formul. Ex. 8 | Initial | 0.08 | 0.11 | 0.34 |
| | Two months of | 0.11 | 0.16 | 1.43 |
| | acceleration | | | |
| | Four months of | 0.15 | 0.21 | 2.50 |
| | acceleration | | | |
| Comp. Formul. | Initial | 0.08 | 0.11 | 0.32 |
| Ex. 4 | Two months of | 0.37 | 0.53 | 1.60 |
| | acceleration | | | |
| | Four months of | 0.89 | 1.27 | 2.31 |
| | acceleration | | | |
| Comp. Formul. | Initial | 0.07 | 0.10 | |
| Ex. 6 | Two months of | 0.09 | 0.13 | |
| | acceleration | | | |
| | Four months of | 0.11 | 0.15 | |
| | acceleration | | | |

As shown in Tables 7 and 8, it was confirmed that the content of salicylic acid increased with times, and there was no significant difference in the increase rate depending on the type of barrier bases. However, the production rates of salicylic acid and the occurrences of atorvastatin lactone and total related compounds were very high in Comparative Formulation Example 7 with no barrier. Hence, it was concluded that it would be preferable to separate said two pharmacologically active ingredients for stability improvement.

Meanwhile, Comparative Formulation Examples 1 and 2 using conventional coating bases, HPMC, and Comparative Formulation Example 3 using an enteric coating base, HPMCP, showed relatively stable appearances, but did not completely prevent the effects of released salicylic acid on an HMG-CoA reductase inhibitor. That is, salicylic acids released from aspirin hinder the stability of atorvastatin, and consequently its stability became poor compared to that of Comparative Formulation Example 5 using only atorvastatin. In particular, it was shown that much amounts of atorvastatin lactone and total related compounds were produced.

In addition, Formulation Examples 1 to 7 containing aspirin coated with EC or carnauba wax as a hydrophobic barrier showed highly improved stability than Comparative Formulation Examples. This indicates that salicylic acid penetrates conventional coating bases such as HPMC or HPMCP to affect an HMG-CoA reductase inhibitor, while it does not penetrate hydrophobic additives such as EC and carnauba wax not to affect the stability of atorvastatin. Such phenomenon was similarly observed in other HMG-CoA reductase inhibitor, rosuvastatin.

Thus, in a complex formulation containing aspirin and an HMG-CoA reductase inhibitor, it is considered that a complex formulation comprising an aspirin granule coated with a hydrophobic additive-containing barrier can provide improved storage stability, such formulation being utilized as a stable and excellent agent for treating hypertension and hypercholesterolemia.

### Experimental Example 2

### Dissolution Test of Aspirin

The aspirin pellets of Examples 2 to 5 and Comparative Examples 1 and 4 were placed into capsules (e.g., gelation capsule; Capsugel) in an amount corresponding to 100 mg of aspirin, respectively, and tested for the dissolution in artificial gastric juice (pH 1.2) and in artificial enteric juice (phosphate buffer, pH 6.8) by 10 rpm according to USP apparatus 1 (basket). The analysis was carried out in accordance with the USP (United States Pharmacopeia) specification for ‘aspirin tablet’ and ‘aspirin delayed-release capsule’. In addition, ‘Aspirin Protect®’ (Bayer, Germany) and ‘Astrix®’ capsule (Boryung, Korea), which are known to be released depending on pH due to their enteric coating, were subjected to the dissolution test in the same methods. The results are shown in Tables 9 and 10.

TABLE 9
Dissolution (%) in pH 6.8

| Time | | | | | Comp. | Comp. | Aspirin | |
|---|---|---|---|---|---|---|---|---|
| (min) | Ex. 2 | Ex. 3 | Ex. 4 | Ex. 5 | Ex. 4 | Ex. 1 | Protect | Astrix |
| 0 | 0.0 | 0.0 | 0.0 | 0.0 | 0.0 | 0.0 | 0.0 | 0.0 |
| 15 | 61.2 | 55.5 | 47.1 | 32.5 | 3.5 | 60.2 | 22.4 | 32.1 |
| 30 | 95.4 | 88.4 | 76.5 | 52.4 | 9.5 | 94.2 | 62.3 | 58.8 |
| 60 | 99.3 | 97.8 | 95.7 | 83.4 | 22.2 | 95.7 | 94.3 | 88.7 |
| 120 | 97.7 | 98.9 | 97.9 | 97.7 | 42.3 | 95.5 | 93.6 | 94.3 |

TABLE 10
Dissolution (%) in pH 1.2

| Time | | | | | Comp. | Comp. | Aspirin | |
|---|---|---|---|---|---|---|---|---|
| (min) | Ex. 2 | Ex. 3 | Ex. 4 | Ex. 5 | Ex. 4 | Ex. 1 | Protect | Astrix |
| 0 | 0.0 | 0.0 | 0.0 | 0.0 | 0.0 | 0.0 | 0.0 | 0.0 |
| 15 | 61.0 | 53.1 | 46.2 | 31.5 | 2.8 | 61.3 | 1.0 | 1.1 |
| 30 | 93.4 | 87.1 | 75.3 | 54.3 | 8.4 | 91.1 | 2.1 | 2.2 |
| 60 | 97.3 | 97.2 | 94.4 | 82.8 | 19.0 | 93.3 | 2.6 | 3.2 |
| 120 | 98.7 | 97.3 | 98.2 | 96.7 | 37.4 | 94.5 | 3.1 | 4.0 |

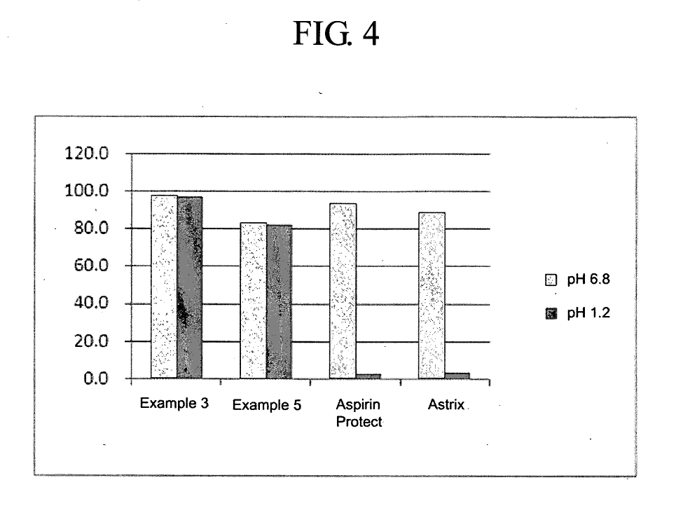

FIG. 4 was plotted based on the results of Tables 9 and 10. As shown in FIG. 4, the dissolution rates of ‘Aspirin Protect®’ and ‘Astrix®’ were significantly changed with pH, whereas the complex formulations of the present invention which contains aspirin coated with a hydrophobic additive-containing barrier showed no changes in dissolution rate with pH.

In addition, the dissolution rates of Examples 2 to 5 and Comparative Example 4 after 1 hour in pH 1.2 slowly decreased with the increase in amounts of hydrophobic additives, and its releases were dramatically delayed, in particular, when the amount of a hydrophobic additive exceeds 60%.

While the invention has been described with respect to the above specific embodiments, it should be recognized that various modifications and changes may be made to the invention by those skilled in the art which also fall within the scope of the invention as defined by the appended claims.

What is claimed is:

1. A complex formulation for the prevention or treatment of cardiovascular diseases, comprising: a) aspirin coated with a barrier containing a hydrophobic additive; and b) an HMG-CoA reductase inhibitor.

2. The complex formulation of claim 1, wherein the amount of the hydrophobic additive is 3.8˜60% by weight based on the total weight of the barrier.

3. The complex formulation of claim 1, wherein the hydrophobic additive is selected from the group consisting of carnauba wax, glyceryl monostearate, glyceryl monooleate, beeswax, ethyl cellulose, aminoalkyl methacrylate copolymer RS, ethyl acrylate-methyl methacrylate copolymer, polyvinyl chloride, polyvinyl acetate, cellulose acetate, and combinations thereof.

4. The complex formulation of claim 1, wherein the HMG-CoA reductase inhibitor is selected from the group consisting of mevastatin, rosuvastatin, atorvastatin, lovastatin, pravastatin, pravastatin lactone, pitavastatin, bervastatin, velostatin, simvastatin, rivastatin, fluvastatin, cerivastatin, and isomers, salts and combinations thereof.

5. The complex formulation of claim 1, wherein the amount of the HMG-CoA reductase inhibitor is 5 to 80 mg.

6. The complex formulation of claim 1, wherein the amount of the aspirin is 10 mg to 2 g.

7. The complex formulation of claim 1, which further comprises a stabilizing agent for the improvement of the stability of HMG-CoA reductase inhibitor.

8. The complex formulation of claim 7, wherein the stabilizing agent is selected from the group consisting of antioxidants, minerals, basic additives, and organic acids and salts thereof.

9. The complex formulation of claim 8, wherein the antioxidant is tocopherol, butylated hydroxytoluene(BHT), butylated hydroxyanisole(BHA), ascorbic acid or erythorbic acid, the mineral is CaCO₃, MgCO₃, NaHCO₃, KH₂PO₄ or K₂HPO₃, the basic additive is meglumine, arginine or glycine, and the organic acid is citric acid or fumaric acid.

10. The complex formulation of claim 1, wherein each of the aspirin and the HMG-CoA reductase is formulated into a pellet or a granule.

11. The complex formulation of claim 1, which further comprises an enteric-coated layer between the aspirin and the barrier.

12. The complex formulation of claim 11, wherein the enteric coating is hydroxypropyl methylcellulose phthalate, cellulose acetate phthalate, methacrylic acid copolymer, or hydroxypropyl methylcellulose acetate succinate.

13. The complex formulation of claim 12, wherein the weight ratio of the enteric coating is 0.1 to 0.5 based on 1 weight of the core.

14. A use of the complex formulation of claim 1 for the manufacture of a medicament for preventing or treating cardiovascular diseases.
